SEQUENCE LISTING (10 sequences)

SEQ ID NO: 1 - protein, 223 aa, Homo sapiens [sequence omitted]

SEQ ID NO: 2 - DNA, 1110 bp, Homo sapiens
  misc_feature (1)..(1110): Human ubiquitin carboxyl-terminal esterase L1
cctgggcggctccgctagctgtttttcgtcttccctaggctatttctgccgggcgctccgcgaagatgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtctgcagagaattcaccgagcgtgagcaaggagaagtccgcttctctgccgtggctctctgcaaggcagcctaatgctctgtgggagggactttgctgatttcccctcttcccttcaacatgaaaatatatacccccccatgcagtctaaaatgcttcagtacttgtgaaacacagctgttcttctgttctgcagacacgccttcccctcagccacacccaggcacttaagcacaagcagagtgcacagctgtccactgggccattgtggtgtgagcttcagatggtgaagcattctccccagtgtatgtcttgtatccgatatctaacgctttaaatggctactttggtttctgtctgtaagttaagaccttggatgtggtttaattgtttgtcctcaaaaggaataaaacttttctgctgataagataaaaaaaaaaaaaaaaaa

SEQ ID NO: 3 - DNA, 21 bp, Artificial Sequence
  shLenti 1.1/scrambl control virus shRNAi sequence
aatcgcatagcgtatgccgtt

SEQ ID NO: 4 - DNA, 72 bp, Artificial Sequence
  shLenti 1.1/UCHL_a - homology to human and mouse sequence
cggaattccccgagatgctgaacaaagttcaagagactttgttcagcatctcgggtttttgatatctagaca

SEQ ID NO: 5 - DNA, 71 bp, Artificial Sequence
  shLenti 1.1/mUCHL_a - homology to mouse sequence
cggaattcagctgaagccgatggagattattcgtaatctccatcggcttcagcttttttgatatctagaca

SEQ ID NO: 6 - DNA, 71 bp, Artificial Sequence
  shLenti 1.1/mUCHL_b - homology to mouse sequence
cggaattccctaaagtttacttcatgaagttcgcttcatgaagtaaactttaggtttttgatatctagaca

SEQ ID NO: 7 - protein, 11 aa, Homo sapiens
  MISC_FEATURE (1)..(11): UCH-L1 N-terminal amino acids
MQLKPMEINPE

SEQ ID NO: 8 - DNA, 19 bp, Homo sapiens
cccgagatgctgaacaaag

SEQ ID NO: 9 - DNA, 21 bp, Mus musculus
agctgaagccgatggagatta

SEQ ID NO: 10 - DNA, 21 bp, Mus musculus
cctaaagtttacttcatgaag

(19) United States
(12) Patent Application Publication
(10) Pub. No.: US 2009/0208508 A1
(43) Pub. Date: Aug. 20, 2009

(54) NOVEL USE OF UBIQUITIN C-TERMINAL HYDROLASE-L1

(75) Inventors: Kong Joo Lee, Seoul (KR); Hyun Jung Kim, Seoul (KR); Jeong Woo Cho, Daejeon (KR); Cheol Young Maeng, Daejeon (KR); You Kyung Nam, Seoul (KR); Sang Mi Lim, Seoul (KR); Young Mee Kim, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: SK CORPORATION, Seoul (KR)

(21) Appl. No.: 12/094,955

(22) PCT Filed: Nov. 24, 2006
(86) PCT No.: PCT/KR06/04989

§ 371 Date: Aug. 22, 2008

(30) Foreign Application Priority Data
Nov. 24, 2005 (KR) 10-2005-0113101

(51) Int. Cl. A61K 39/395; A61K 31/7105; C12Q 1/68

(52) U.S. Cl. 424/141.1; 530/300; 424/130.1; 435/6; 514/44 A

(57) ABSTRACT

The present invention relates to a composition for diagnosing cancer metastasis comprising Ubiquitin C-terminal hydrolase-L1 (UCH-L1), a use of UCH-L1 for diagnosing cancer metastasis and a method for diagnosing cancer metastasis using the same; a composition for suppressing cancer metastasis comprising an inhibitor of UCH-L1, a use of an inhibitor of UCH-L1 for suppressing cancer metastasis and a method of suppressing cancer metastasis using the same; a composition for screening a cancer metastasis inhibitor comprising UCH-L1 protein, a use of UCH-L1 protein for screening a cancer metastasis inhibitor and a method of screening cancer metastasis inhibitor using the same; a composition for screening a cancer metastasis inhibitor comprising UCH-L1 gene, a use of UCH-L1 gene for screening a cancer metastasis inhibitor, a method of screening cancer metastasis inhibitor using the same.

UCH-L1 is a key molecule to modulate cell migration including the cancer invasion according to their expressing level. Thus, the monoclonal and polyclonal antibodies and the substrate of UCH-L1 can be used for diagnosis of cancer metastasis.

Also, the cancer metastasis can be suppressed by inhibiting of the expression of UCH-L1 or activity of the enzyme, thus we can screen and develop an inhibitor of UCH-L1 and use it as an adjuvant drug for anticancer therapy.

### TECHNICAL FIELD

The present invention relates to a composition for diagnosing cancer metastasis comprising Ubiquitin C-terminal hydrolase-L1 (UCH-L1), use of UCH-L1 for diagnosing cancer metastasis and a method for diagnosing cancer metastasis using the same; a composition for suppressing cancer metastasis comprising an inhibitor of UCH-L1, use of an inhibitor of UCH-L1 for suppressing cancer metastasis and a method of suppressing cancer metastasis using the same; a composition for screening a cancer metastasis inhibitor comprising UCH-L1 protein, use of UCH-L1 protein for screening a cancer metastasis inhibitor, and a method of screening cancer metastasis inhibitor using the same; a composition for screening a cancer metastasis inhibitor comprising UCH-L1 gene, use of UCH-L1 gene for screening a cancer metastasis inhibitor, a method of screening cancer metastasis inhibitor using the same.

### BACKGROUND ART

Ubiquitin-C-terminal hydrolase-L1 (UCH-L1) is one of deubiquitinating enzymes (DUBs) and a neuron-specific ubiquitin recycling enzyme.

It was reported that UCH-L1 hydrolyses the peptide bond between ubiquitin and small adduct or unfolded polypeptide in vitro. However, the substrate specificity of UCH-L1 enzyme and specific characteristics of the isoforms of DUBs family are poorly understood [Christopher N. et al., “substrate specificity of deubiquitinating enzymes; Ubiquitin C-terminal hydrolase,” Biochemistry, 37, 3358-3368, 1998].

UCH-L1 is an ubiquitin hydrolase widely expressed in neuronal tissues at all stages of neuronal differentiation and is specifically expressed in neurons and cells of diffuse neuroendocrine system and their tumors. UCH-L1 is expressed in most parts of cerebrum, especially highly in substantia nigra of mesencephalon. DUBs are subdivided into ubiquitin C-terminal hydrolase (UCHs) and ubiquitin processing protease (UBPs) or ubiquitin-specific proteases (USPs) according to their sequence homology. Despite the high-sequence homology, the in vitro hydrolytic activities of these two enzymes do not differ significantly.

UCH enzymes contain 4 conserved sequence motifs usually spanning a domain of approximately 200 amino acids and UCH is smaller in size than UBP. One of the conserved motifs contains a critical cysteine in the active site as well as a oxyanion hole residue glutamine. The other encodes histidine and aspartic acid residues present in the active site. The residues act together in a catalytic mechanism similar to that found in other cysteine proteases such as papain/cathepsin B [Simon S. Wing. Deubiquitinating enzymes—the importance of driving in reverse along the ubiquitin-proteasome pathway, The International Journal of Biochemistry & Cell Biology, 35, 590-605, 2003].

It was reported that UCH-L1 is a major target of oxidative damage in AD and PD brains and is extensively modified by carbonyl formation, methionine oxidation, and cysteine oxidation (J Biol. Chem. 2004, Mar. 26; 279(13):13256-64, Oxidative modifications and down-regulation of ubiquitin carboxyl-terminal hydrolase L1 associated with idiopathic Parkinson's and Alzheimer's diseases). Furthermore, it was reported that UCH-L1 is a biomarker which is highly expressed in various cancers, for example; lung cancer, colorectal cancer, and pancreatic cancer, myeloma [Otsuki T, et al., “Expression of protein gene product 9.5 (PGP9.5)/ubiquitin-C-terminal hydrolase 1 (UCH-L1) in human myeloma cells”, Br J. Heamatol., Nov; 127(3):292-8, 2004; Guoan Chen et al., “Proteomic analysis of lung adenocarcinoma: Identification of highly expressed set of proteins in tumor”, Clinical Cancer Research, Vol. 8, 2298-2305, 2002; Taiji Yamazaki et al., “PGP9.5 as a Marker for invasive colorectal cancer”, Clinical Cancer Research, vol. 8, 192-195, 2002; Hidefumi Sasaki, et al., “Expression of the protein gene product 9.5, PGP9.5, is correlated with T-status in non-small cell lung cancer”, Jpn J Clin Oncol., 31(11), 532-535, 2001].

The expression of UCH-L1 is specifically associated with lung cancer development and is abundantly present in both small-cell lung cancer (SCLC) and non-small-cell lung cancer (NSCLC) cell lines regardless of hASH1 status [Kenji Hibi, et al., “PGP9.5 As a Candidate Tumor Marker for Non-Small-Cell Lung Cancer” American Journal of Pathology, Vol. 155, No. 3, pp 711-715, 1999]. UCH-L1 is preferentially expressed in T3/T4 NSCLC compared with T1/T2 NSCLC [Hidefumi Sasaki, et al., “Expression of the protein gene product 9.5, PGP9.5, is correlated with T-status in non-small cell lung cancer”, Jpn J Clin Oncol., 31(11) 532-535, 2001]. In patient with lung cancer, circulating UCH-L1 antigen and/or antibodies were detected in sera [Franck Brichory, et al., “Proteomics-based Identification of Protein Gene Product 9.5 as a Tumor Antigen That Induces a Humoral Immune Response in Lung Cancer”, CANCER RESEARCH, Vol. 61, pp 7908-7912, 2001].

US Patent Publication No. 2005-79560 (publication date: 2005 Apr. 14) discloses a composition and a method for treating and diagnosing colon cancer using UCH-L3 which is an isoform of UCH-L1 and a cancer marker. This publication details the fact that UCH-L3 is a cancer antigen expressed highly in colon tumor 3-5 times more than in other tumor forms, and a diagnosing and analyzing method for examining the expression of UCH-L3, and provides an antibody for UCH-L3 and describes a method for cancer immune therapy using UCH-L3.

As stated above, it has been reported that UCH-L1 and isoforms of UCH-L1 are cancer markers. However, it is reported here for the first time that UCH-L1 is related to the invasion required for cancer metastasis.

We found that UCH-L1 is specifically related to cancer metastasis or invasion using 2D gel electrophoresis and proteomic analysis and demonstrated that UCH-L1 is a key target molecule in cancer invasion.

### DISCLOSURE OF INVENTION

### Technical Problem

An object of the present invention is to provide a composition for diagnosing cancer metastasis comprising Ubiquitin C-terminal hydrolase-L1 (UCH-L1), an antibody or a substrate for UCH-L1, or a primer or probe for UCH-L1 gene, use of UCH-L1 for diagnosing cancer metastasis, and a method of diagnosing cancer metastasis using the same.

Also, another object of the present invention is to provide a composition for suppressing cancer metastasis comprising an inhibitor of UCH-L1, use of an inhibitor of UCH-L1 for suppressing cancer metastasis and a method for suppressing cancer metastasis using the same.

Yet another object of the present invention is to provide a composition for screening a cancer metastasis inhibitor comprising UCH-L1 protein, use of UCH-L1 protein for screening a cancer metastasis inhibitor, a method of screening cancer metastasis inhibitor using the same, a composition for screening a cancer metastasis inhibitor comprising UCH-L1 gene, use of UCH-L1 gene for screening a cancer metastasis inhibitor, and a method of screening cancer metastasis inhibitor using the same.

### Technical Solution

The present invention provides a composition for diagnosing cancer metastasis comprising Ubiquitin C-terminal hydrolase-L1 (UCH-L1), use of UCH-L1 for diagnosing cancer metastasis and a method of diagnosing cancer metastasis using the same.

The Ubiquitin C-terminal hydrolase-L1, UCH-L1 comprises the amino acid sequence of SEQ ID No.: 1 (NCBI accession No.: P09936), which is encoded by the base sequence comprising the sequence of SEQ ID No.: 2.

The expression level of UCH-L1 is specifically increased in cancer cells with metastatic property, and as the expression of UCH-L1 is increased, cell migration is also increased.

Thus, the diagnosis of cancer metastasis can be performed by quantitative analysis of the expression of UCH-L1 in cancer cells or tissues.

The present invention also provides a method for diagnosing cancer metastasis comprising the following steps of: (a) obtaining protein samples expressed from tissues or cells of mammals; and (b) examining the existence of UCH-L1 in the obtained sample and performing quantitative analysis.

The present invention also provides a composition for diagnosing cancer metastasis comprising a monoclonal antibody for UCH-L1 protein having the amino acids sequence of SEQ ID No.: 1.

Also, the present invention provides a method for diagnosing cancer metastasis comprising the steps of: (a) obtaining protein samples expressed from tissues or cells of mammals; (b) reacting the obtained samples with a monoclonal antibody for UCH-L1, thereby examining the existence of UCH-L1 in the obtained sample and performing quantitative analysis.

The diagnosis may be performed by using the color reaction between the composition for diagnosing cancer metastasis comprising a monoclonal antibody for UCH-L1 and the sample materials obtained from a living body (cancer cells or protein extract of tissues or serum, etc.) using ELISA (Enzyme Linked Immunosorbent assay), and quantitative analysis. In addition, the cancer metastasis may be diagnosed by using UCH-L1 specific histoimmunostaining for cancer tissues or cells obtained from a living body, or comparing and analyzing the UCH-L1 expression level in cancer tissues or cells obtained from a living body with that of normal group through western blot analysis.

For example, the diagnosis of cancer metastasis may be also performed by the following steps of; (1) preparing immuno-affinity column by adhering the monoclonal antibody for UCH-L1 to gel support; (2) measuring the amount of UCH-L1 in the material obtained from a living body (cancer cells or protein extract of tissues or serum, etc.) by HPLC using the immuno-affinity column of the step 1; (3) comparing and analyzing the quantitative analysis data.

The monoclonal antibody for UCH-L1 may be prepared according to common monoclonal antibody preparation processes, or obtained commercially.

Generally, the monoclonal antibody for UCH-L1 can be analyzed quantitatively by color reaction using a second antibody conjugated with enzymes such as alkaline phosphatase (AP) or horseradish peroxidase (HRP) etc. and substrates thereof. Otherwise, UCH-L1 monoclonal antibody directly conjugated with AP or HRP etc. can be used for the quantitative analysis.

Instead of the UCH-L1 monoclonal antibody, a polyclonal antibody recognizing UCH-L1 protein may also be used. The polyclonal antibody may be prepared by usual antiserum preparation processes.

The present invention also provides a composition for diagnosing cancer metastasis comprising a polyclonal antibody for 11 amino acids located in N-terminal region of UCH-L1. Instead of the UCH-L1 monoclonal antibody, a polyclonal antibody recognizing II amino acids (SEQ ID. No.: 7) in N-terminal region of UCH-L1 protein may be used. The polyclonal antibody may be prepared by published methods available in literature.

Also, the present invention provides a composition for diagnosing cancer metastasis comprising a substrate for UCH-L1 having the amino acid sequence of SEQ ID No.: 1.

The substrate for the UCH-L1 may be ubiquitinylated substances. Preferably, UB-AMC (ubiquitinylated amino-4-methylcoumarin) may be used.

The UCH-L1 enzyme can be quantitatively analyzed by detecting the fluorescence at the time of hydrolysis of the UB-AMC by UCH-L1.

The diagnosis may be performed by adding the composition for diagnosing cancer metastasis to the sample material obtained from a living body (cancer cells or protein extract of tissues), thereby detecting the luminous reaction and analyzing the data.

Also, the present invention provides a method for diagnosing cancer metastasis comprising the following steps of: (a) obtaining protein samples expressed from tissues or cells of mammals; (b) reacting the obtained samples with a substrate for UCH-L1, detecting the fluorescence, thereby examining the expression of UCH-L1 and performing quantitative analysis.

The present invention also provides a composition for diagnosing cancer metastasis comprising a PCR primer or a probe for UCH-L1 gene having the base sequence of SEQ ID No.: 2.

Cancer metastasis can be diagnosed by comparing the expression level of UCH-L1 between test group and normal group through RT-PCR or quantitative RT-PCR using the primer for UCH-L1 gene. Also, cancer metastasis can be diagnosed by comparing the expression levels of UCH-L1 between test group and normal group through Northern blot analysis etc. using the probe for the UCH-L1 gene.

Cancer metastasis can be diagnosed by reacting the material obtained from a living body (RNA extracted from cancer cells or tissues), and comparing the expression level of UCH-L1 between test group and normal group (through RT-PCR, quantitative RT-PCR, or Northern blotting).

Also, the present invention provides a method for diagnosing cancer metastasis by: (a) obtaining RNA samples expressed from tissues or cells of mammals; and (b) reacting the obtained samples with a primer or a probe for the UCH-L1, thereby examining the expression of UCH-L1 in the obtained sample and performing quantitative analysis.

The present invention provides a composition for suppressing cancer metastasis comprising an inhibitor of UCH-L1, use of an inhibitor of UCH-L1 for suppressing cancer metastasis, and a method for suppressing cancer metastasis using the same.

Inhibition of cancer metastasis by the present invention includes alleviation and prevention of cancer metastasis.

The present composition can be used for inhibition of cancer metastasis by administering the UCH-L1 expression inhibitor to patients, since UCH-L1 is highly expressed in cancer cells.

The present invention also provides a method for suppressing cancer metastasis which comprises administering an inhibitor of UCH-L1 to cancer patients as a therapeutically effective amount to suppress a cancer metastasis.

The UCH-L1 inhibitor may be the materials repressing the expression of UCH-L1 or inhibiting the enzymatic activity, specifically, shRNAi (short hairpin RNAi); isatin oximes compounds (Chem. Biol., 10(9):837-846, 2003) known as a UCH-L1 inhibitor in the prior art; or monoclonal antibody for UCH-L1 protein, or polyclonal antibody for 11 amino acids in the N-terminal region of UCH-L1.

FIG. 13 is a schematic diagram of lentiviruses used in preparation of shRNAi of UCH-L1.

In the present invention, the shRNAi of UCH-L 1 is preferably one or more selected from those of SEQ ID Nos.: 8, 9 and 10.

The base sequences of SEQ ID No.: 3 to 6 are prepared for the cases that lentiviral vector be used as an expression vector.

The SEQ ID No.: 3 is a base sequence of lentivirus control shRNAi comprising scramble sequence.

The SEQ ID No.: 4 is shRNAi prepared for targeting a common base sequence region at shRNAi of human and mouse, and used for inducing a knock-down of UCH-L1 using a lentiviruses as an expression vector (chemistry and biology. vol 10. 837-846, 2003).

The SEQ ID Nos.: 5 and 6 are shRNAi prepared for targeting a base sequence region at mouse by using lentiviruses as an expression vector.

The SEQ ID No.: 8 is an important target sequence capable of application in all various siRNA (small interference RNA), for lowering UCH-L1 expression in human and mouse.

The SEQ ID Nos.: 9 and 10 are important target sequences capable of application in all various siRNA (small interference RNA), for lowering UCH-L1 expression in mouse.

Through preliminary experiments, it was found that the expression level of UCH-L1 is effectively reduced in siRNA using a region of SEQ ID No.: 8 on human-base sequences, and in siRNA using a region of SEQ ID No.: 8 or 10 on mouse-base sequences. A base sequences of human and mouse show the amino acid sequence homology of 95% on clustalW multiple sequence alignment program. When lentivirus is used as expression vector, coexpression with GFP or zeosin resistance gene can confirm whether the cells are infected by lentivirus (Nature genetics, vol. 33, March 2003).

In one embodiment of the present invention, shRNAi inhibiting an expression of UCH-L1 is expressed in melanoma cell of mouse to cause a RNA interference (RNAi), resulting in inhibiting the expression of UCH-L1. Microinjecting into vein of a mouse with the said melanoma cell shows that the primary metastasis into lung is reduced.

The composition for suppressing cancer metastasis of the present invention may also comprise one or more anticancer drugs. The inhibitor or antibody of UCH-L1 protein may be used with chemotherapeutic agents which are well-known to a skilled artisan, for example, alkylating agents such as cyclophosphamide, aziridine, alkylalconsulphonate, nitrosourea, dacarbazine, carboplatin, cisplatin, etc., antibiotics such as mitomycin C, anthracycline, doxorubicin (adriamycin) etc., antimetabolitic agents such as methotrexate, 5-fluorouracil, cytarabine etc., plant-derived agents such as Vinka alkaloid, and hormone etc.

The composition of the present invention comprising an inhibitor of UCH-L1 as an active ingredient may be prepared by using pharmaceutically and physiologically acceptable additives besides the active ingredient. Such additives may be excipient, disintegrating agent, sweetener, binder, coating agent, blowing agent, lubricant, glidant, flavoring agent, solubilizer, etc.

The composition of the present invention comprising an inhibitor of UCH-L1 as an active ingredient may further comprise one or more pharmaceutically acceptable carriers besides the active ingredient, to be formulated to pharmaceutical composition appropriately.

In order to formulate liquid formulation, the composition comprise the pharmaceutically acceptable carriers which have biocompatibility and are sterilized, for example, saline, sterilized water, Ringer's solution, buffered saline, albumin injection solution, dextrose solution, malto dextrin solution, glycerol, ethanol and a mixture thereof. If necessary, other common additives such as antioxidant, buffer, bacteriostatic agent, etc. may be added in the composition. Also, by further adding a diluent, dispersing agent, surfactant, binder or lubricant, the composition may be formulated to injectable forms such as solution, suspension, emulsion, etc., pill, capsule, granule or tablet. Furthermore, the present composition may be formulated in any desirable forms according to disease or ingredient, by using conventional methods or the written text of Remington's Pharmaceutical Science (Mack Publishing Company, Easton Pa.).

The pharmaceutical composition of the present invention may be prepared in any form such as granule, powder, coated tablet, tablet, capsule, suppository, syrup, juice, suspension, emulsion, drop or injectable liquid formulation, and sustained release formulation of the active ingredient(s), etc.

The composition of the present invention comprising an inhibitor of UCH-L1 as an active ingredient may be administered via various routes including intravenous, intra-arterial, intraperitoneal, intramuscular, intrathoracic, transdermal, intranasal, inhalation, topical, rectal, oral, ocular and intradermal introduction, according to conventional method of administration.

The dosage of the present composition comprising an inhibitor of UCH-L1 as an active ingredient represents an amount of active ingredient or pharmaceutical composition that is required to induce suppression effect of cancer metastasis.

Thus, the dosage can be determined by various factors such as the nature of the disease, the severity of patient's symptom, the content of active ingredient, the nature and content of other ingredients, the type of formulation, patient's age, body weight, health status, gender and food, dosing time, administration route, the secretion ratio of composition, time of treatment, and other co-administrated drug. In adults, when an inhibitor of UCH-L 1 is administrated one or more times per day, the dosage is preferably 0.01 ng/kg˜10 mg/kg in case of shRNAi, 0.1 ng/kg˜10 mg/kg in case of compounds, 0.1 ng/kg˜10 mg/kg in case of UCH-L1 monoclonal antibody.

The present invention provides a composition for screening a cancer metastasis inhibitor comprising a UCH-L1 gene, and a use of a UCH-L1 gene for screening a cancer metastasis inhibitor, and a method for screening a cancer metastasis inhibitor using the same.

A UCH-L1 gene sequence of the composition according to the present invention may be one more selected from a part or whole base sequences of SEQ ID No.: 2; and other base sequences comprising a polymorphism of SEQ ID No.: 2. Furthermore, the composition for screening a cancer metastasis inhibitor may comprise the UCH-L1 gene sequence as a form being included in a expression vector system of prokaryote or eukaryote.

The present invention also provides a method for screening a cancer metastasis inhibitor using the composition for screening a cancer metastasis inhibitor comprising a UCH-L1 gene.

The method for screening a cancer metastasis inhibitor according to the present invention may comprise the steps of: contacting the composition for screening a cancer metastasis inhibitor comprising a UCH-L1 gene with the test material, then examining their reaction, and deciding whether the test material suppresses the gene expression.

To confirm the reaction between the composition comprising a UCH-L1 gene and the test material in the screening method according to the present invention, it is possible to use conventional methods used in conformation of reaction among the cases of DNA-DNA, DNA-RNA, DNA-protein or DNA-compound, for example, a method for measuring an expression rate of the gene by hybridization detection for binding between the gene and the test material in vitro; northern analysis, quantitative PCR, quantitative real time-PCR etc. after reacting mammal cells with the subject material; or a method for measuring an expression rate of a reporter protein by reacting with the test material after joining the reporter gene to the said UCH-L1 gene to induce into cell.

Besides the UCH-L1 gene, the composition of the present invention can comprise distilled water or buffer to maintain a structure of nucleic acid stably.

The present invention provides a composition for screening a cancer metastasis inhibitor comprising a UCH-L1 protein, and a use of UCH-L1 protein for screening a cancer metastasis inhibitor, and a method for screening a cancer metastasis inhibitor using the same.

A UCH-L1 protein sequence of the composition according to the present invention may be one more selected from a part or whole base sequences of SEQ ID No.: 1; and the amino acid sequence of the protein expressed from base sequences comprising a polymorphism of SEQ ID No.: 2, and polypeptide fragment of UCH-L1 with equal physiological activity.

Since the composition for screening according to the present invention has an effect on facilitation of cancer metastasis, it is possible to screen and select the materials inhibiting a transcription into mRNA or translation into protein in UCH-L1 gene sequence, or inhibiting an activity of UCH-L1 protein as a cancer metastasis inhibitor.

The present invention also provides a method for screening a cancer metastasis inhibitor using the composition for screening a cancer metastasis inhibitor comprising a UCH-L1 gene as target materials.

The method for screening a cancer metastasis inhibitor according to the present invention comprises the following steps of: contacting the composition for screening a cancer metastasis inhibitor comprising a UCH-L1 protein with the test material, examining their reaction and deciding whether the test material enhances or suppresses a function of the protein comprised in the composition.

In the method for screening according to the present invention, for confirming the reaction between composition comprising a UCH-L1 protein and the subject materials, it is possible to use conventional methods used in the cases of DNA-DNA, DNA-RNA, DNA-protein or DNA-compound, for example, a method of measuring an activity after reacting UCH-L1 gene or UCH-L1 protein with the subject material, a method of yeast two-hybrid, a searching for phage-displayed peptide clone bound with UCH-L1 protein, high throughput screening (HTS) using natural substance and chemical library and the like, drug hit HTS, cell-based screening and DNA array.

Besides the protein expressed form UCH-L1, the composition of the present invention can comprise a buffer or reaction solution to maintain a structure or physiologic activity of protein stably.

Moreover, the composition of the present invention can comprise a cell expressing the UCH-L1 protein, or cell containing a plasmid expressed under promoter capable of controlling a transcription rate, and the like.

In the method for screening according to the present invention, the test material could be one from an individual nucleic acid, protein, any extracts, natural substances or compounds, which are assumed to have a possibility of cancer metastasis inhibitor, or chosen randomly among them.

In FIG. 18 of the present invention, the specific example of the method for screening an inhibitor using UCH-L1 is described. 5˜10 nM of UCH-L1 enzyme is reacted with 0˜1,000 nM of UB-AMC (substrate) and reaction buffer (50 mM Tris-HCl (pH 7.6), 0.5 mM EDTA, 0.1 mg/mL albumin, W/O or W/5 mM DTT), the candidate material of UCH-L1 inhibitor are added thereto simultaneously, and analysis of biochemical characteristic such as enzyme activity, enzyme reaction rate and HTS analysis are performed to screen a UCH-L1 inhibitor.

In the test material obtained by method for screening according to the present invention, which is for enhancing or inhibiting an expression of gene or function of the protein, the materials of the former case can be candidate cancer metastasis inhibitors. In the materials of the latter case, it is possible to discover a candidate material of cancer metastasis inhibitor by developing an inhibitor about the materials inhibiting an expression of gene or function of the protein.

Henceforth, such candidate materials of cancer metastasis inhibitor will act as leading compound in the development of cancer metastasis inhibitor, and the leading compound can be used in modifying or optimizing the structures of the UCH-L1 gene or UCH-L1 protein in order to inhibit their functions, thereby developing a new cancer metastasis inhibitor.

Since the substances obtained by this method have an effect to UCH-L1 gene or UCH-L1 protein partially or completely, it is possible that the cancer metastasis and other diseases caused by inhibiting of UCH-L1 gene expression or lowering a function of UCH-L1 protein are suppressed.

The matters relating to genetic engineering technologies will be more explicit from the written text described in Sambrook, et al. Molecular Cloning, A Laboratory Manual, Cold Spring Harbor laboratory Press, Cold Spring Harbor, N.Y. (2001) and Frederick M. Ausubel et al., Current protocols in molecular biology volume 1, 2, 3, John Wiley & Sons, Inc. (1994).

The advantages and features of the present invention and the method of revealing them will be explicit from the following examples described in detail. However, it is to be distinctly understood that the present invention is not limited thereto but may be otherwise variously embodied and practiced. It is obvious that the following examples are to complete the disclosure of the invention and to indicate the scope of the present invention to a skilled artisan completely, and the present invention will be defined only by the scope of the claims.

### ADVANTAGEOUS EFFECTS

UCH-L1 is a key molecule to modulate cell migration including the cancer invasion according to their expression level. Thus, the monoclonal and polyclonal antibodies and the substrate of UCH-L1 can be used for diagnosis of cancer metastasis.

Also, cancer metastasis can be suppressed by inhibiting of the expression of UCH-L1 or activity of the enzyme, thus we can screen and develop an inhibitor of UCH-L1 and use it as an adjuvant drug for anticancer therapy.

### DESCRIPTION OF DRAWINGS

FIG. 1 shows the schematic diagram for the whole process to identify the target proteins related to invasion in two cell lines having different invasion potentials.

FIG. 2 shows the results from invasion assay of WI38, H358, and H157 cell lines having different invasion potentials using the transwell coated with Matrigel™.

FIG. 3 shows the results from gelatinase activity assay of WI38, H358 and H157 cell lines having different invasion potentials.

FIG. 4 shows proteomic analysis of intra or extra cellular proteins of H358 and H157 cancer cell lines having different invasion potentials using the 2D gel electrophoresis.

FIG. 5 shows the relative expression level of intra or extra cellular proteins of WI38, H358 and H157 cell lines.

FIG. 6 shows the result from the invasion assay of WI38 normal lung cell line transiently transfected with pFlag-CMV-2-UCH-L1.

FIG. 7 shows the result from the invasion assay of HeLa cervical cancer cell line, in which UCH-L1 is not expressed, transiently transfected with pFlag-CMV-2-UCH-L1

FIG. 8 shows the proteomic analysis of HEK 293T cell lines transiently transfected with pFlag-CMV-2-UCH-L1 to identify the binding proteins to UCH-L1 using immunoprecipitation.

FIG. 9 shows the Western blotting analysis with anti-beta actin, anti-HSP70, and anti-ERK antibodies to identify UCH-L1 binding proteins as shown in FIG. 8 and Table 3.

FIG. 10 shows the immunofluorescent analysis of the cellular colocalization of flag-tagged UCH-L1 and beta-actin in the cell membrane of H358 cancer cell line using confocal microscopy.

FIG. 11 shows the schematic diagram of pulmonary metastasis assay (animal experiment).

FIG. 12 shows the protein expression level of UCH-L1 in B 16F 10 mouse melanoma cell line and H157 human lung cancer cell line using the Western blotting analysis.

FIG. 13 shows the schematic diagram of lentivirus-based system to prepare the shRNAi of UCH-L1.

FIG. 14 shows the Western blotting analysis with anti-UCH-L1 antibody to confirm the UCH-L1 expression reduced in H157 stable cells expressing UCH-L1 specific RNAi.

FIG. 15 shows the results from the FACS analysis to measure the GFP expression level in H157 stable cell expressing UCH-L1 specific RNAi using FACS flow cytometry.

FIG. 16 shows the images of lungs with or without the black spherical colonies extirpated from BALB/C nude mouse injected with the B16F10 stable cells expressing irrelevant RNAi or UCH-L1 specific RNAi and being selected with Zeocin antibiotics (on 14th day after the injection).

FIG. 17 shows the number of the black spherical colonies on the same lung surface shown in FIG. 16, which were counted by using dissecting microscope.

FIGS. 18 and 19 show the schematic diagram of high-throughput screening (HTS) for inhibitors of UCH-L1 enzymatic activity.

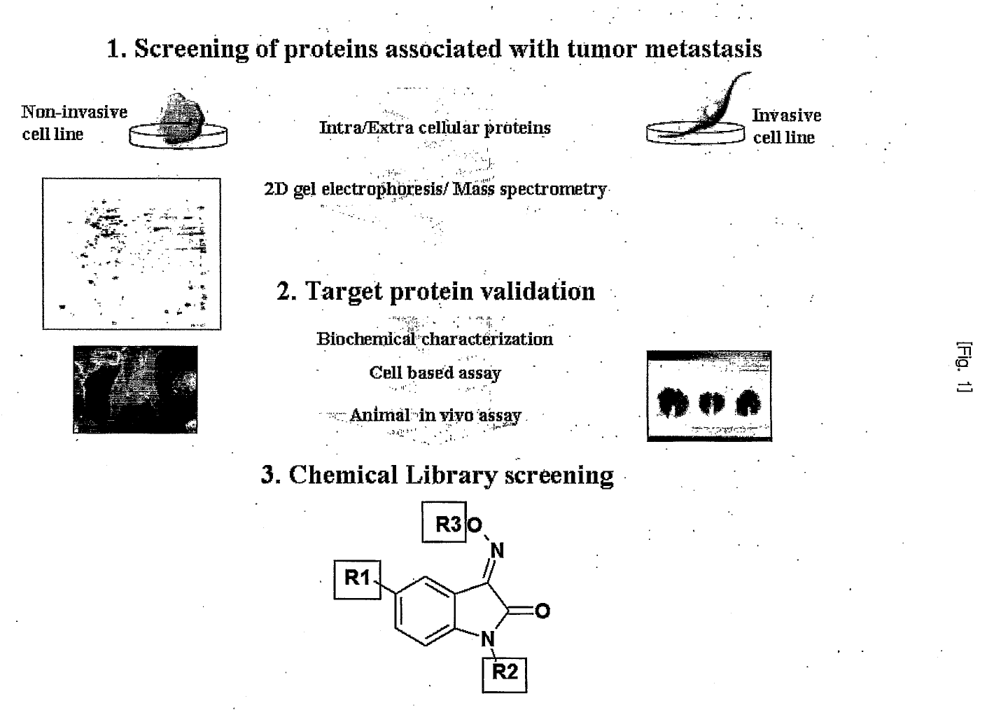

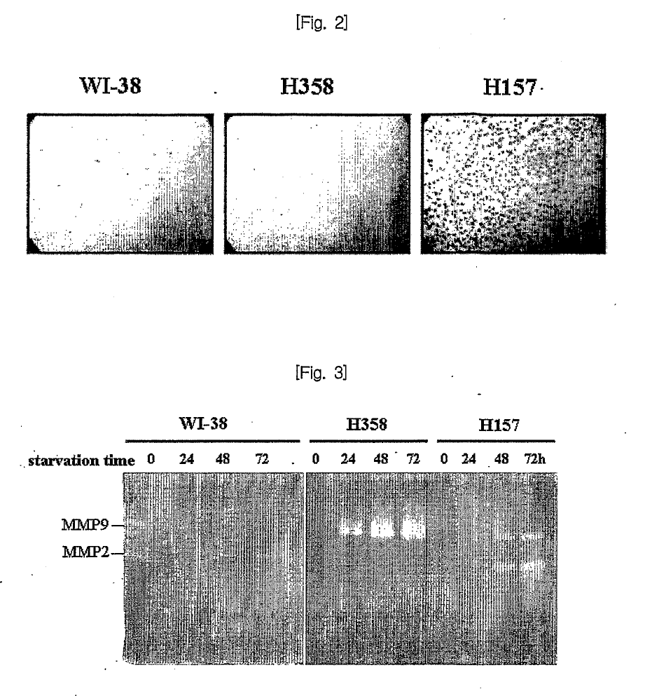

### MODE OF INVENTION

### Example I

### Diagnosis for Cancer Metastasis, Suppression of Cancer Metastasis and Screening of Inhibitors for Cancer Metastasis Using UCH-L1

I-1. Screening of Proteins Associated with Cancer Metastasis to Identify a Target Molecule

To identify the proteins related to the cancer metastasis, we analyzed the proteins differentially expressed in intra or extra cellular fractions of H157 (squamous cell carcinoma) having the invasion potential, H358 (bronchi alveolar carcinoma) cancer not having the invasion potential, and WI38 (VA-13 subclone 2RA (fibroblast)) as a normal lung cell line.

H157, H358, and WI38 cells were maintained in RPMI-1640 (pH 7.3 with 2 mM L-glutamine) supplemented with 10% (v/v) fetal bovine serum (FBS), 100 unit/mL penicillin, 100 μg/mL, streptomycin, 3.75 μg/mL sodium bicarbonate, and 10 mM HEPES.

I-1-1) Invasion Assay Using the Transwell Coated with Matrigel™

To examine the cell migration capability, the invasion assay in vitro using the transwell coated with Matrigel™ (Matrigel, Becton Dickinson (BD) company, Cat No. 354234) which is an reconstituted basement membrane matrix extracted from EHS (Englebreth-Holm-Swarm) mouse tumor was performed.

Matrigel™ was diluted with serum free cold media to 1 mg/mL (final concentration) at 4ε. The upper chamber of transwell invasion chamber was coated with the above diluted Matrigel™ (80 μg/μL) and then incubated in 37□ for 1 h. The lower chamber of transwell invasion chamber was incubated for equilibration with 600 μL of RPMI-1640 supplemented with 10% FBS. After 1 h, uncoated Matrigel™ was removed from the transwell with serum free media.

H157, WI38, and H358 cells were harvested with trypsin, then suspended with serum free media, and same amount of cells (1.0×10⁴ cells/well, 150 μL) were respectively seeded on the upper chamber of transwell coated with Matrigel™ and incubated at 37□ for 24 h. After incubating and removing the residual cells in upper chamber of transwell with cotton balls, the number of cells migrated toward lower chamber of transwell was counted using the hemocytometer after staining the cells with crystal violet (crystal violet 2.5 g, methanol 125 mL, d-H₂O 375 mL) for 5 min and washing three times with distilled water for 1 min. Data were shown as the mean number of cell migrated □ S.D. from three independent experiments.

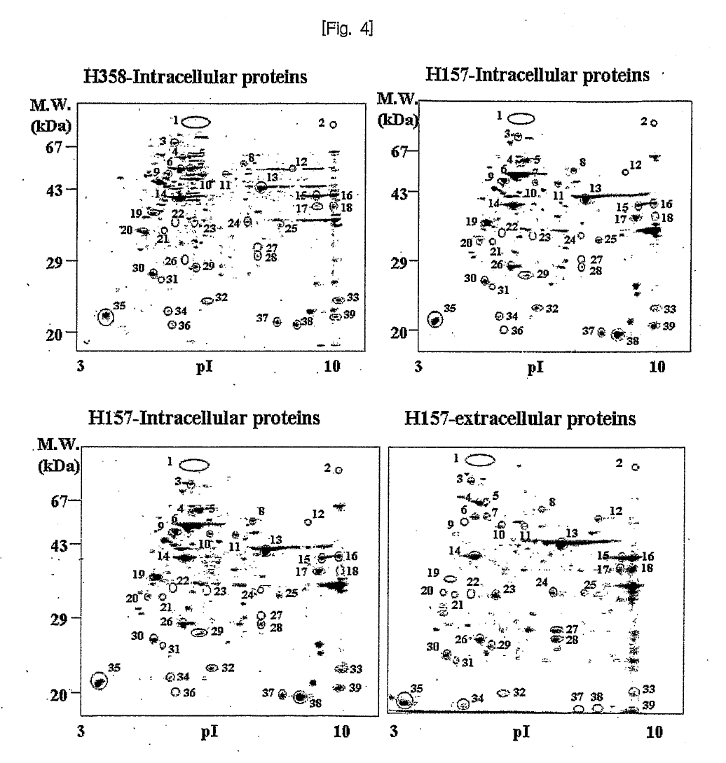

As shown in FIG. 2, it was found that H157 cell has the most invasive potential in the above three cell lines, WI38 cells is next, and H358 cells has no invasion potential although it belongs to lung cancer cells.

I-1-2) The Activity Assay of MMP2 and MMP9 Using the Zymography

To investigate the relationship between cell migration and the expression of MMPs in various cancer cell lines, the activity of MMPs was measured using the Zymography.

Zymography is the method to assay the activity of gelatinase. After the proteins were separated on the SDS-PAGE including the gelatin, the gel was incubated at 37□ for 15-20 h. The bands having the activated MMPs (gelatinases) on the incubated gel were not stained with coomassie blue dye because the gelatin was hydrolyzed by the activated MMPs (gelatinases). These bands are called as negative bands.

To investigate the activities of MMPs in WI38, H358, and H157 cell lines, the proteins of the cell lines were extracted, and then Zymography was performed according to the above procedure.

As shown in FIG. 3, WI-38 has the activity of MMP 2 and H358 has the activity of MMP 9. However, H157 has the activities of MMP 2 and MMP 9.

As shown in FIGS. 2 and 3, it was suggested that there is the correlation between the invasive potential and the activities of MMPs. That is, H157 cells which has the activities of MMP2 and MMP9 at the same time has the highly invasive potential. On the other hand, the invasive potentials were low in the H358 and WI38 cells, which have the only one of MMP activities. H358 cells having the activity of MMP9 was not invasive and WI38 cells having the activity of MMP2 was less invasive than H157 cells.

Metastasis is the process by which tumors spread from the primary organ in which they arose to other sites in the body. In this process, tumor cells must be able to invade across a barrier called the basement membrane and gelatinases like MMPs must activate to invade the barrier. Based on these processes and the above invasion assay results, it was suggested that MMP2 activity is required for the migration and invasion of cancer cell and MMP9 has the additive effect on the migration and invasion of cancer cell by MMP2. It was found that the cell migration and invasion in cancer are not regulated by only MMP9.

I-1-3) 2D-Gel Electrophoresis and MALDI-TOF Mass Spectrometry

To identify the differentially expressed proteins in various cell lines, 2D-gel electrophoresis was performed.

2D-PAGE was performed using strip gels obtained from Amersham Pharmacia Biotech (USA). Proteins were isoelectrically focused on strip gel according to their pI values and then separated on SDS-PAGE with their molecular weights.

H358 and H157 cells (1□10⁶ cells: 150-200 μg) were lysed for 1 h at room temperature with a first dimensional sample buffer (30 μL) containing 8.4 M urea, 2.4 M thiourea, 5% CHAPS, 50 mM DTT, 1.6% 5-7 ampholyte, 0.4% 3-10 ampholyte, 1 mM PMSF, 5 μg/mL aprotinin, 10 μg/mL pepstatin A, 10 μg/mL leupeptin, and 1 mM EDTA. To separate the cell lysate according to pI, the rehydration buffer (125 μL; 8 M urea, 2% (w/v) CHAPS, 0.5% IPG buffer, 1% bromophenol blue) was added to strip holder. The lysed cell samples were added thereto, and then the strip gel was put thereon. In order to prevent evaporation of moisture, 180 μL of mineral oil was added, and the strips were rehydrated for more than 12 h at 20□ and then electrofocused. The following focusing protocol was used: 1) 500 V for 1 h; 2) 1000 V for 1 h; and 3) 8000 V for 3 h. After electrofocusing, the strips were stored in −70° C. rapidly to prevent separated proteins from being diffused.

For separation of proteins by molecular weight (second separation), the strip gel was shaken for 20 min with equilibration buffer (50 mM Tris-HCl [pH 8.8], 6 M urea, 30% (v/v) glycerol, and 2% (w/v) SDS, 1% bromophenol blue, 100 mM DTT) and loaded and separated onto 11% SDS-PAGE.

The differentially expressed proteins (spots) in three cell lines (H157, H358, and WI38) to be identified by 2D-PAGE (the above method) were shown in FIG. 4.

For the analysis using MALDI-TOF mass spectrometry, the proteins were separated on 2D-PAGE and then stained with coomassie dye. After being identified, the proteins (spots) on gels were excised and stored in eppendorf tubes. The excised gels were homogenized in 30% methanol (1 mL) and then incubated at 37° C. for 5 min. These gels were destained three times with 100 μL of 25 mM ammonium bicarbonate (NH₄HCO₃)/50% acetonitrile solution for 10 min, dehydrated with 100% acetonitrile, and completely dried using Speed Vac vacuum centrifuge. The trypsin (10 μL, 10 ng/mL) solution including 25 mM NH₄HCO₃ was added to the dried gels and reacted with them at room temperature for 5-10 min and successively at 4° C. for 30 min. Next, 20 μL of 25 mM NH₄HCO₃ was added thereto. These gels treated with trypsin and 25 mM NH₄HCO₃ (20 μL) were incubated at 37° C. for 12-16 h additionally. Here, 30 μL of 60% acetonitrile/0.1% TFA (1:1 v/v) solution was added. These gels were incubated at 37° C. for 10 min. The supernatants were put together three times, and then were treated with 20 μL of 100% acetonitrile, finally dried in SpeedVac vacuum centrifuge, and stored in −70° C.

The concentrated and dried samples were dissolved in 10 μL of 60% acetonitrile/0.1% TFA (1:1 v/v) solution and sonicated for 2 min. Next, these were mixed with the matrix solution (α-cyano-4-hydroxycinnamic acid in acetonitrile/0.1% TFA (1:1 v/v) in ratio of 1:1, loaded on the palate, and dried at room temperature. Mass analysis was performed with HP G2025A MALDI-TOF MS (Hewlett-Packard, Palo Alto, USA) equipped with a nitrogen laser (337 nm). The accelerated voltage from ion source was 28 kV, spectra was quantitated using external substrate P (MH⁺=1348.6 Da) and insulin (MH⁺=5778.6 Da). Data were collected and identified with the Internet protein resource (UCSF) “MS-Fit” (http://prospector.ucsf.edu/) program to analyze the spectra. Image Master 2D gel analysis software (Amersham Pharmacia Biotech., USA) was used for quantity.

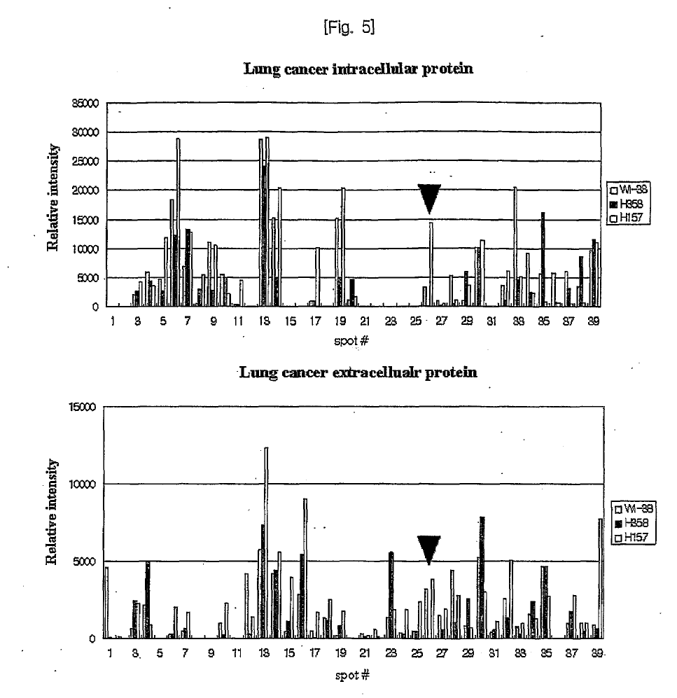

The differentially expressed proteins in various cell lines using the above methods were shown in Table 1 and FIG. 5.

UCH-L1 is one of the most differentially expressed proteins in H157 cell line in comparison with H358 lung cancer cell line. UCH-L1 is detected in both of intra- and extra-cellular proteins of H157 cells using proteomic analysis.

TABLE 1

| | Intracellular protein | NCBI # | Extracellular protein |
|---|---|---|---|
| 1 | | 4755885 | Pro alpha 1 (1) collagen |
| 2 | | 1418930 | Prepte-alpha 2 (1) collagen |
| 3 | Transitional endoplasmic reticulum ATPase | 5410290 | Transitional endoplasmic reticulum ATPase |
| 4 | 71 Kd heat shock cognate protein | 32467 | 71 Kd heat shock cognate protein |
| 5 | Mitochondrial stress-70 protein precursor | P38646 | Mitochondrial stress-70 protein precursor |
| 6 | Heavy metal bindg protein | 34707/306890 | Chaperonin (HSP 60) |
| 7 | T-complex protein (TCP) | 1351211/603955 | KIAA0098 protein |
| 8 | Transformation sensitive protein IEF SSP | P31948 | |
| 9 | Vimentin | 2119204 | |
| 10 | Protein disulfide isomerase (PDI) | 1085373 | Protein disulfide isomerase (PDI) |
| 11 | T-complex protein 1 | P78371 | |
| 12 | Pyruvate kinase | 266427 | Pyruvate kinase |
| 13 | Alpha-emolase | 4503571/1167848 | Alpha-enolase |
| 14 | Actin | 1070613/28252 | Beta-actin |
| 15 | Phosphoglycerate kinase | 387020 | Phosphoglycerate kinase |
| 16 | Phosphoglycerate kinase | 387020 | Phosphoglycerate kinase |
| 17 | Aldolase A | 28614 | Aldolase A |
| 18 | Aldolase A | 28614 | Aldolase A |
| 19 | B23 nucleophosmin (280AA) | 825671 | B23 nucleophosmin (280AA) |
| 20 | Similar to trophmyosin 4 | 12653955 | |
| 21 | | 4505751 | Lipcortin V |
| 22 | | 7106894 | HSPC252 |
| 23 | | 6434861 | Phosphatidylcholome transfer protein |
| 24 | | 2217923 | Alpha (1,2) fucosyltrasnferase |
| 25 | | 4757756 | Lipcortin II |
| 26 | Similar to ubiquitin carboxy-terminal hydrolase L1 | 12653131 | Similar to ubiquitin carboxy-terminal hydrolase L1 |
| 27 | Phosphoglycerate mutase 2 | 450571 | Phosphoglycerate mutase 2 |
| 28 | Triosphosphate isomerase | 37247 | Triosphosphate isomerase |
| 29 | Gluththione S-transferase (GST-Pi) | 31946 | Gluththione S-transferase (GST-Pi) |
| 30 | Tropomyosin | P07226/37424 | Cytoskeletal tropomyosin |
| 31 | | 339958 | Skeletal tropomyosin |
| 32 | Nm23H1g | 468542 | Nm23H1g |
| 33 | cyclophilin | 181250 | cyclophilin |
| 34 | Imitiation factor 4D | 181997/4691541 | adeylate kinase 5 |
| 35 | Calmodulin | 179810 | Calmodulin |
| 36 | Retionic acid binding protein II | 181026 | |
| 37 | Peptidyl isomerase | 30168 | Peptidyl isomerase |
| 38 | Peptidyl isomerase | 30168 | Peptidyl isomerase |
| 39 | Nm23H2 product | 189240 | Nm23H2 product |

The above results suggested a possibility that UCH-L1 may be used a protein closely related to cancer metastasis.

### I-2. Identification of UCH-L1 as a Target Molecule to Regulate Cancer Metastasis

I-2-1) Invasion Assay of WI38 Cell Line Exclusively Expressed with Flag-Tagged UCH-L1

To investigate whether UCH-L1 is a target molecule to regulate the cancer cell invasion, we examined the invasion capability of normal lung cell line WI38 that was exclusively expressed with flag-tagged UCH-L1 [pFlag-CMV-2-UCH-L1, pFlag-CMV-2 expression vector (sigma, saint louis, USA) including the sequence no. 2 of UCH-L1 full length cDNA] in a dose dependent manner (1, 2, 4, 6 μg), using the transwell coated with Matrigel™ (invasion assay).

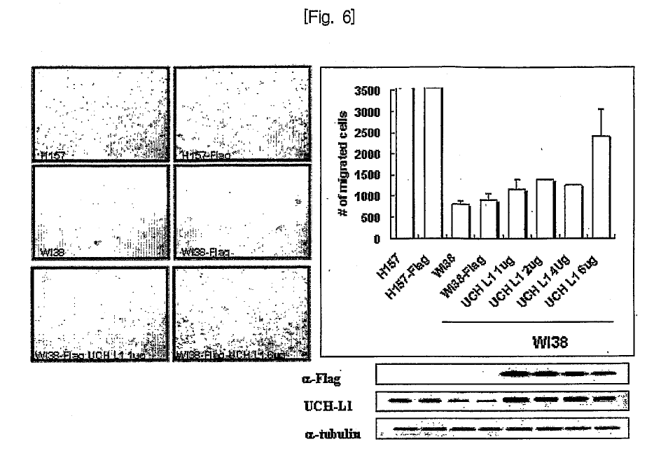

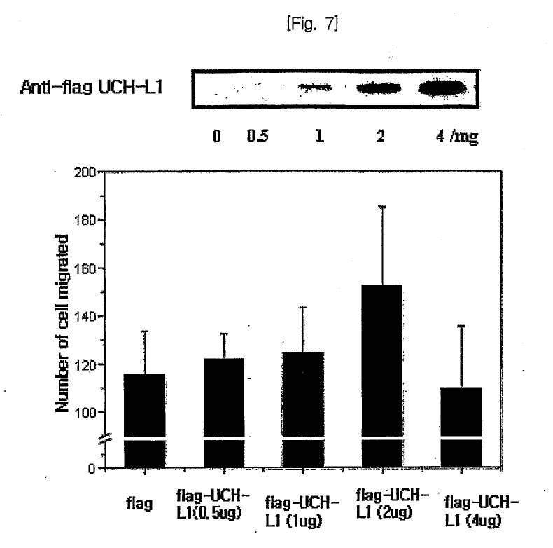

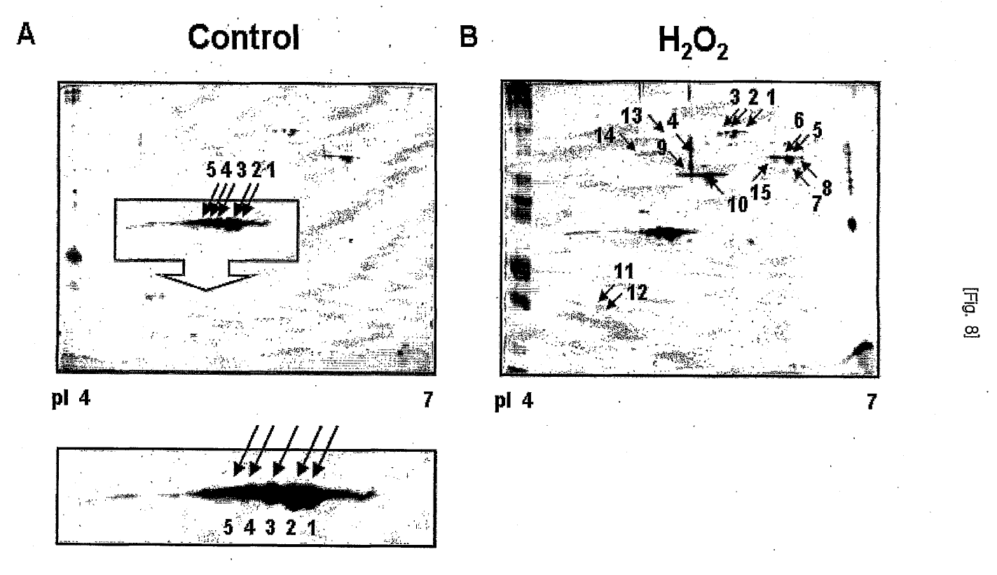

H157 cells with superior cell migration capacity were used as a positive control in this invasion assay. As shown in FIG. 6, the transfection with pFlag-CMV-2-UCH-L1 makes increase the cell migration capacity. Specifically, WI38 cells over-expressing flag-tagged UCH-L1 significantly increased cell migration in a dose dependent manner.

I-2-2) Invasion Assay of HeLa Cells Exclusively Expressed with Flag-Tagged UCH-L1

UCH-L1 is not expressed in HeLa cells although they are invasive. After HeLa cells were transiently transfected with pFlag-CMV-2-UCH-L1 in a dose dependent manner (0.5, 1, 2, and 4 μg), it was investigated whether the migration capability of HeLa cells is increased or not.

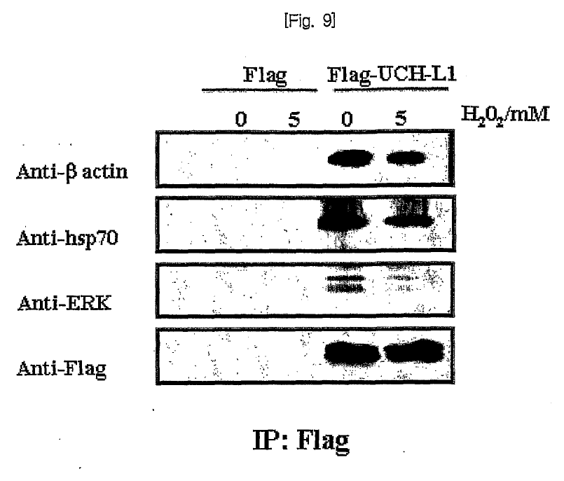

As shown in FIG. 7, the migration of HeLa cells over-expressing UCH-L1 was significantly increased in a dose dependent manner in comparison with that of control cells.

This result suggested that UCH-L1 specifically regulates the cell migration and plays a role in cancer cell invasion.

### I-2-3) Identification of Interacting Proteins of UCH-L1

A. Identification of Interacting Proteins of UCH-L1 Using Immunoprecipitation

The above results suggested that UCH-L1 is a key molecule to regulate cell migration. To identify the cellular interacting proteins of UCH-L1, immunoprecipitation was performed.

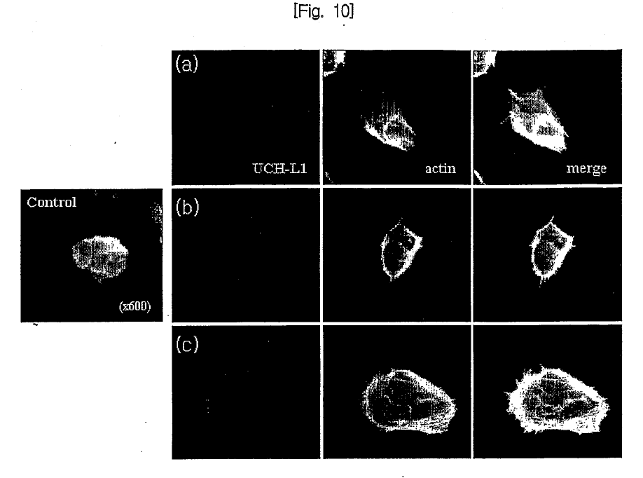

After HEK293T cells were transiently transfected with pFlag-CMV-2-UCH-L1, the interacting proteins of UCH-L1 were screened by using immunoprecipitation. This result was shown in FIG. 8. It was known that there are various isoforms of UCH-L1 in cells by detecting of 5 spots (1-5 spots) having the different motilities according to their pI values in HEK293 cells (FIG. 8A).

To examine the interacting protein profiles of various UCH-L1 isoforms respectively according to oxidative stress, HEK293 cells was treated with H₂O₂. The H₂O₂ treated cell lysates were immunoprecipitated and analyzed by 2D-PAGE (FIG. 8B) and MALDI-TOF MS in comparison with them of the control cells.

Table 2 shows the proteins identified as interacting proteins with UCH-L1 using immunoprecipitation and MALDI-TOF MS.

As shown in Table 2, beta-actin, HSP70, ERK, etc. were identified as UCH-L1 interacting proteins in cells. It suggested that UCH-L1 may be involved in the regulation of cell migration through interacting with the proteins such as Beta-actin and ERK which were known as a protein related to cell morphology.

TABLE 2
Identification of UCH-L1 interaction proteins (MALDI-TOF-MS)

| | | | Mass | | % Co | |
|---|---|---|---|---|---|---|
| Spot | Identified protein | accession# | (Da) | pl | v | Function |
| 1 | heat shock 70 kDa protein 98 precursor; heat shock 70 kD protein 9 | 24234688M | 73681 | 5.9 | 34 | Chaperone |
| 2 | heat shock 70 kDa protein 98 precursor; heat shock 70 kD protein 9 | 24234688M | 73681 | 5.9 | 20 | Chaperone |
| 3 | heat shock 70 kDa protein 8 isoform 1; heat shock cognate protein, 71- | 5729877 M | 70899 | 5.4 | 28 | Chaperone |
| | kDa; heat shock 70 kd protein 10; heat shock cognate protein 54; | | | | | |
| | constitutive heat shock protein 70; lipopolysaccharide-associated protein | | | | | |
| | 1; LPS-associated protein 1[Homo sapiens] | | | | | |
| 4 | keratin 1, type II, cytoskeletal | 7428712 | 65494 | 6.0 | 33 | Cytosketetal |
| | | | | | | protein |
| 5 | heterogeneous nuclear ribonucleoprotein H2; heterogeneous nuclear | 9624998 M | 49264 | 5.9 | 50 | Ribonucleo |
| | ribonucleoprotein H1 [Homo sapiens] | | | | | Protein |
| 6 | heterogeneous nuclear ribonucleoprotein H1 [Homo sapiens] | 5031753 M | 49230 | 5.9 | 25 | Ribonucleo |
| | | | | | | Protein |
| 7 | heterogeneous nuclear ribonucleoprotein H1 [Homo sapiens] | 5031753 M | 49230 | 5.9 | 20 | Ribonucleo |
| | | | | | | Protein |
| | 44 kDa protein kinase, mitogen-activated protein kinase | 1335009 M | 41001 | 6.3 | 23 | signal-regulated |
| | | | | | | kinase |
| 8 | murine mammary tumor integration site 6 (oncogene homolog); | 4503521 M | 52221 | 5.7 | 19 | translation |
| | eukaryotic translation initiation factor 3, subunit 6 (48 kD) [Homo sapiens] | | | | | factor |
| 9 | beta actin; beta cytoskeletal actin | 4501885 M | 41737 | 5.3 | 28 | Cytosketetal |
| | | | | | | protein |
| 10 | actin, beta | 14250401 | 41005 | 5.6 | 27 | Cytosketetal |
| | | | | | | protein |
| 11 | BPNT1 protein | 17389533 | 35772 | 5.5 | 18 | Esterase |
| 12 | ACTB protein | 15277503 | 40221 | 5.6 | 27 | structural |
| | | | | | | protein |

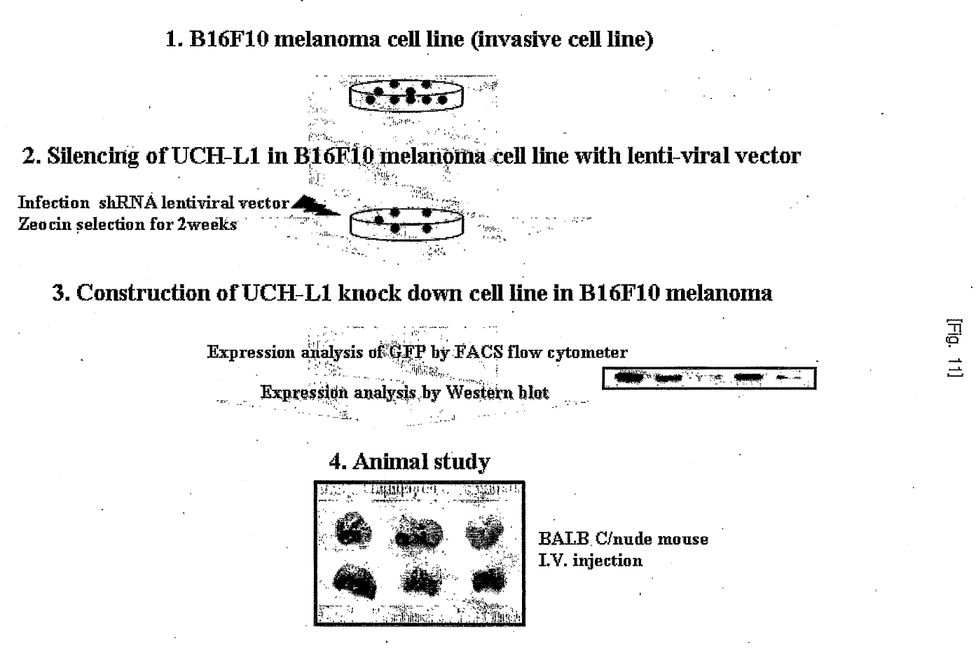

To confirm the interacting proteins shown in Table 2, beta-actin, HSP70, and ERK among them were identified as interacting proteins again using Western blotting with their antibodies respectively (FIG. 9).

B. The Interaction of UCH-L1 and Beta-Actin in Cells

Cell morphological change is deeply related to the cell migration. Beta-actin is involved in the cell morphology. When H358 cells with little UCH-L1 were transiently transfected with pFlag-CMV-2-UCH-L1 to overexpress UCH-L1, its morphology was changed remarkably, and this morphological change was closely associated with cell migration capacity. To confirm the relationship between UCH-L1 and cell migration capacity, subcellular distribution of the UCH-L1 and beta-actin was examined in H358 cells having little UCH-L1.

H358 cells were transiently transfected with pFlag-CMV-2-UCH-L1 and then incubated with phalloidin Fluorescein isothio-cyanate for actin staining, then with anti-flag antibody and then with Texas red conjugated second antibody for UCH-L1 staining.

It was demonstrated that UCH-L1 interacted with beta-actin in cells using the confocal microscopy as shown FIG. 10. Specifically, UCH-L1 is colocalized with beta-actin mainly in cell membrane, not in whole cytoplasm. The results suggested that UCH-L1 is involved in modulation of cell morphology affecting cell migration capacity.

Based on the above results, it was demonstrated that UCH-L1 is a target protein related to cell migration required for cancer metastasis.

### Example II

### Decrease of Cell Migration in B16F10 Mouse Melanoma Stable Cells Expressing UCH-L1 Specific RNAi

We tried to elucidate whether UCH-L1 is a key target molecule related to cancer metastasis through the experimental metastasis using animal model. Normal B16F10 mouse melanoma cells are highly invasive and its first target organ in metastatic process is lung. Using lentivirus-based system, we prepared the B16F10 stable cells in which UCH-L1 was knock-downed.

After preparing B16F10 stable cells, we examined whether the metastasis to lung was decreased when the B16F 10 stable cells were injected intravenously into the tail vein.

FIG. 11 shows the schematic diagram for experimental metastasis using animal model.

II-1. Preparation of B16F10 Mouse Melanoma Stable Cells Expressing UCH-L1 Specific RNAi

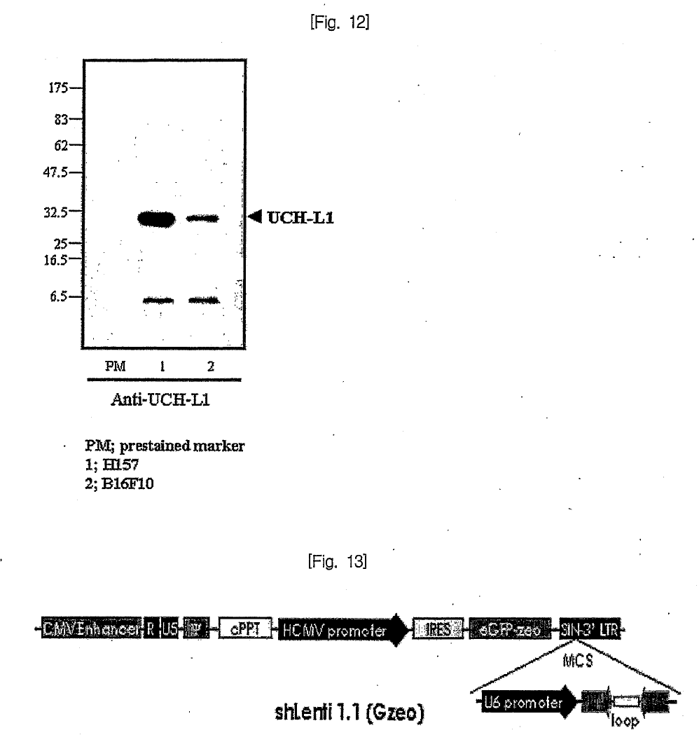

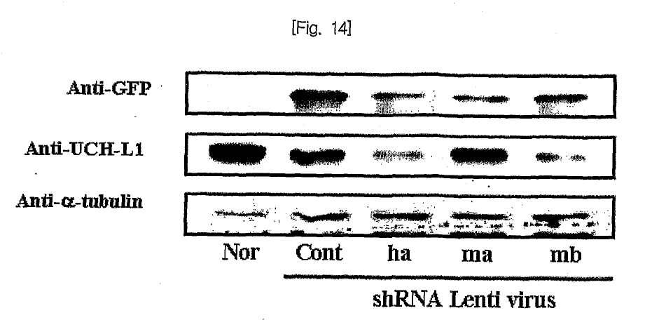

The expression level of UCH-L1 in B16F10 mouse melanoma cell line was analyzed by using the Western blotting with anti-UCH-L1 antibody in comparison with that of H157 lung cancer cell line overexpressing UCH-L1. This result was shown in FIG. 12. It was determined that UCH-L1 is expressed also in B16F10 cell lines.

B16F10 cells (2.5×10⁵ cells, 70˜80% confluency) were seeded on the cell culture dish. After 24 h, these cells were infected with 1 mL of lentivirus (VCA™, Vectorcorea, Inc., Seoul, Korea) expressing irrelevant RNAi or UCH-L1 specific RNAi. 500 μL ( [symbol] ) (virus titer 1□10⁷ IFU/mL) of lentivirus expressing the sequence specific shRNAi (SEQ ID Nos. 3, 8, 9, and 10) was used for infection of 2.5×10⁵ of B16F10 cells.

Polyburene (4˜8 mg/mL) was added thereto, then the cell culture dish was shaken slowly up and down. After incubation for 6 h at 37□ under 5% CO₂, the virus soup was changed with the RPMI-1640 supplemented with 10% FBS. B16F10 cells were infected at twice with lentivirus on 24^{th} hour after the first infection. However the incubation time for second infection was 12 h, not 6 h. After 24 h from the second infection, B16F 10 cells were harvested with trypsin and subcultured on the other dish by diluting the cells with RPMI-1640 (1/16 dilution).

After 24 h, cells were maintained with the cell culture media with 5 μg/mL of Zeocin. For about 14 days, the cell culture media was changed with fresh media containing same amount of Zeocin every three days. On 14th day after start of selection with Zeocin, only cells having the resistance to Zeocin were survival and these cells were infected with lentivirus stably expressing UCH-L1 specific RNAi (shRNAi, short hairpin RNA). On 14th day after start of selection with Zeocin, the colony was formed by the cells having the resistance to Zeocin. The colonized cells were selected and re-subcultured with fresh media. From this method, we could prepare UCH-L1 expression reduced stable cell lines.

As shown in FIG. 14, the UCH-L1 expression level in stable cell lines expressing UCH-L1 specific RNAi was confirmed by Western blotting analysis.

The expression of UCH-L1 was reduced in the B16F10 stable cells (ha and mb) expressing UCH-L1 specific RNAi (SEQ ID No. 8 for ha and SEQ ID No. 10 for mb) in comparison with that of normal B16F10 cells and B16F10 stable cells expressing irrelevant RNAi (control, SEQ ID No. 3). However, the UCH-L1 expression was not reduced in the B16F10 stable cells expressing UCH-L1 specific RNAi, ma (SEQ ID No. 9 for ma). The lentivirus infection and RNAi expression were confirmed by confocal microscopy analysis for detection of co-expressed GFP.

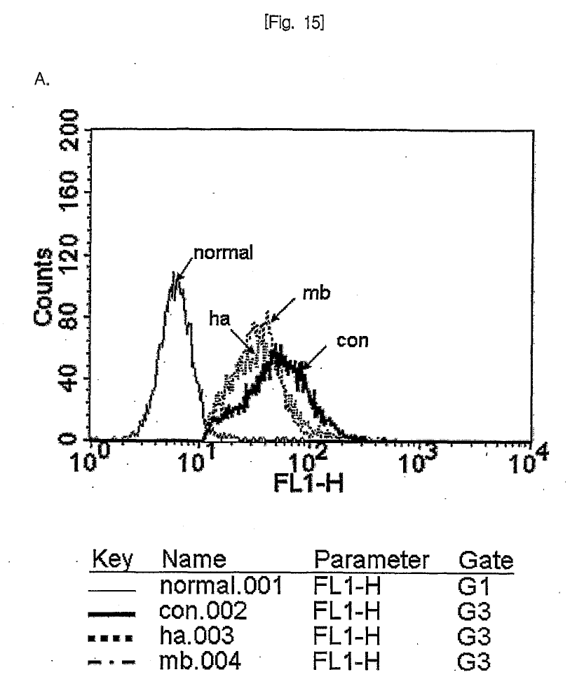

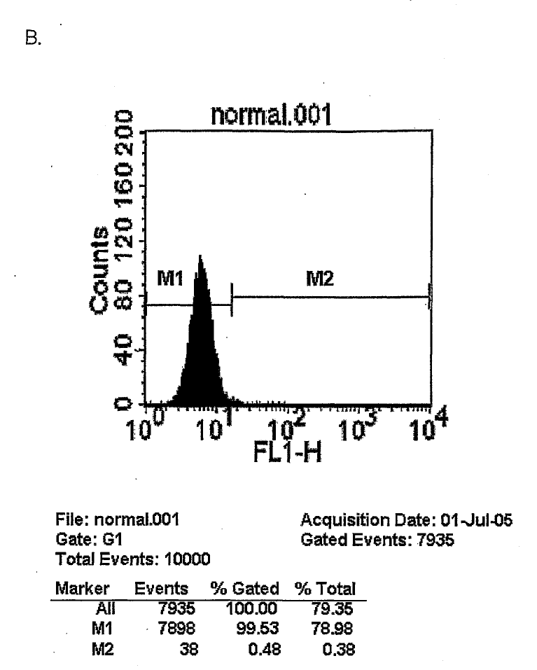

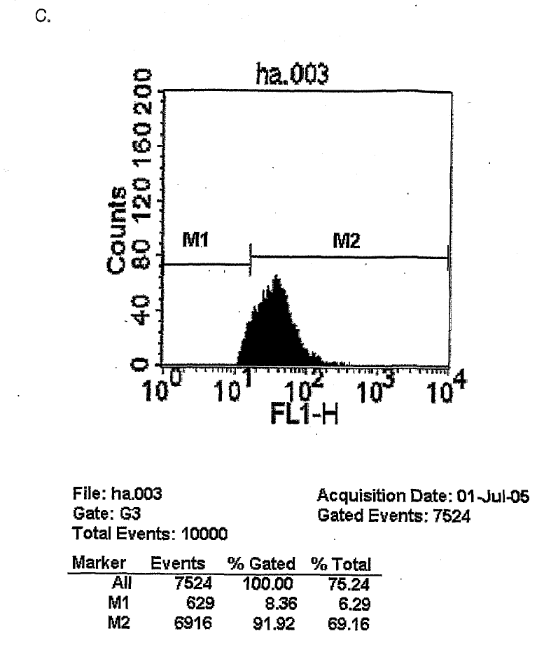

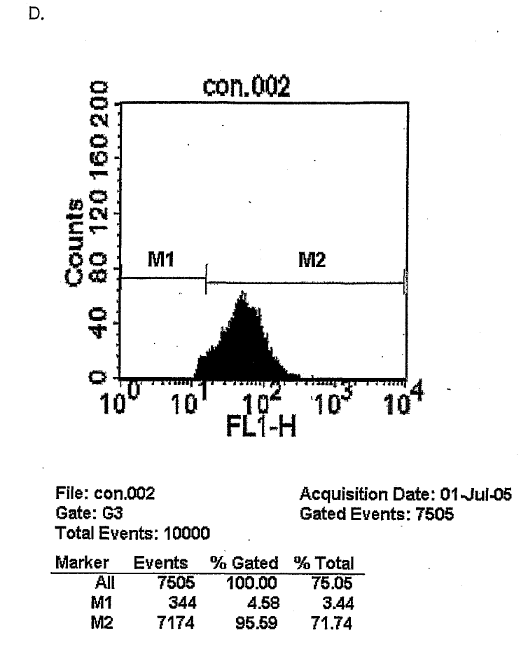

FIG. 15 shows that the GFP expressed by lentivirus infection in stable cells expressing UCH-L1 specific RNAi was confirmed by using FACS flow cytometry. Using the expression level of GFP, it was demonstrated that the stable cells expressing specific sequence RNAi (ha, ma, and mb) was infected with same amount of lentivirus. Therefore, two stable cell lines (ha and mb) were selected for animal experimental metastasis. This animal experimental metastasis was performed with ha and mb stable cell lines in comparison with that using the stable cell lines expressing irrelevant RNAi (control).

II-2. Animal Experiment for Cancer Metastasis Using B16F10 Stable Cells Expressing UCH-L1 Specific RNAi

As stated above, after the UCH-L1 expression knock-downed B 16F 10 stable cells were selected with Zeocin, these cells (1□10⁶ cells) were single-injected into the tail vein of BALB/C nude mouse. On 14th day after injection, the metastasis to lung was investigated. (For control set, BALB/C mouse were injected with same amount of B16F10 stable cells expressing irrelevant RNAi (control lentiviral vector))

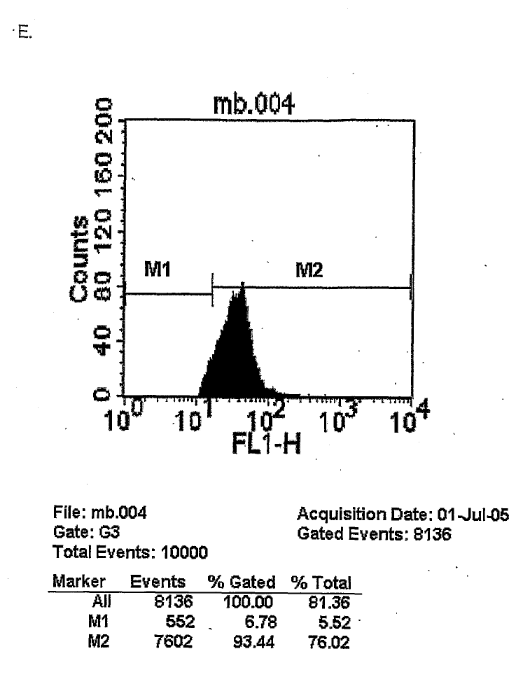

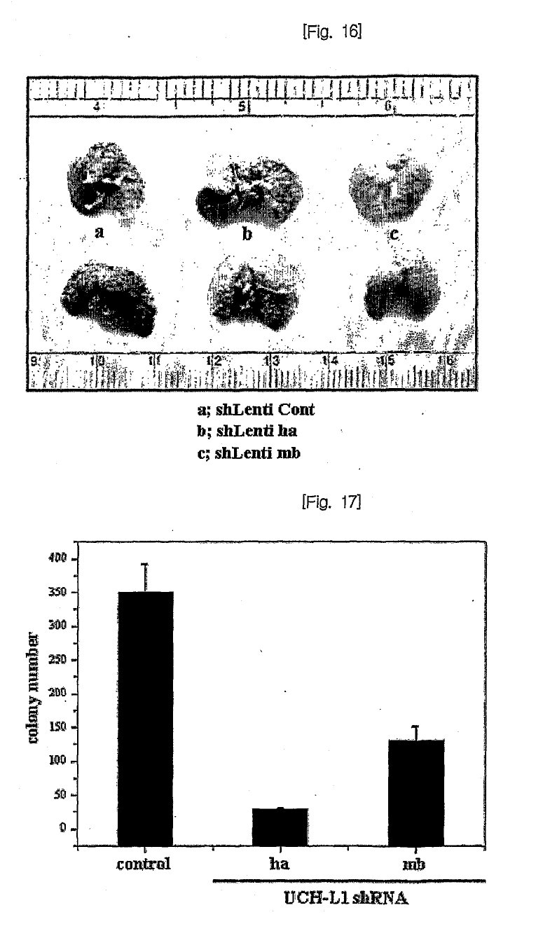

FIG. 16 shows the images of lung on 14th day after injection.

As shown in FIG. 16, the number of colony on the lung excised from the BALB/C nude mouse (b and c) injected with the UCH-L1 expression knock-downed cells was dramatically decreased in comparison with that on the lung excised from the control BALB/C mouse (a).

FIG. 17 shows the quantitative results of the black spherical colonies on the same lung surface shown in FIG. 16 using dissecting microscopy. These result suggested that the metastasis to lung is dramatically reduced in B16F10 stable cells expressing UCH-L1 specific RNAi by lentivirus infection in comparison with control B16F10 cells having the metastatic potential (92% reduction for ha and 62% reduction for mb).

These findings suggested that UCH-L1 is a key regulator for cell migration required for cancer metastasis, suggesting that the suppression of UCH-L1 is a possible way to inhibit the cancer invasion and metastasis.

### Example III

### The Screening of the Inhibitors for Cancer Metastasis Using UCH-L1

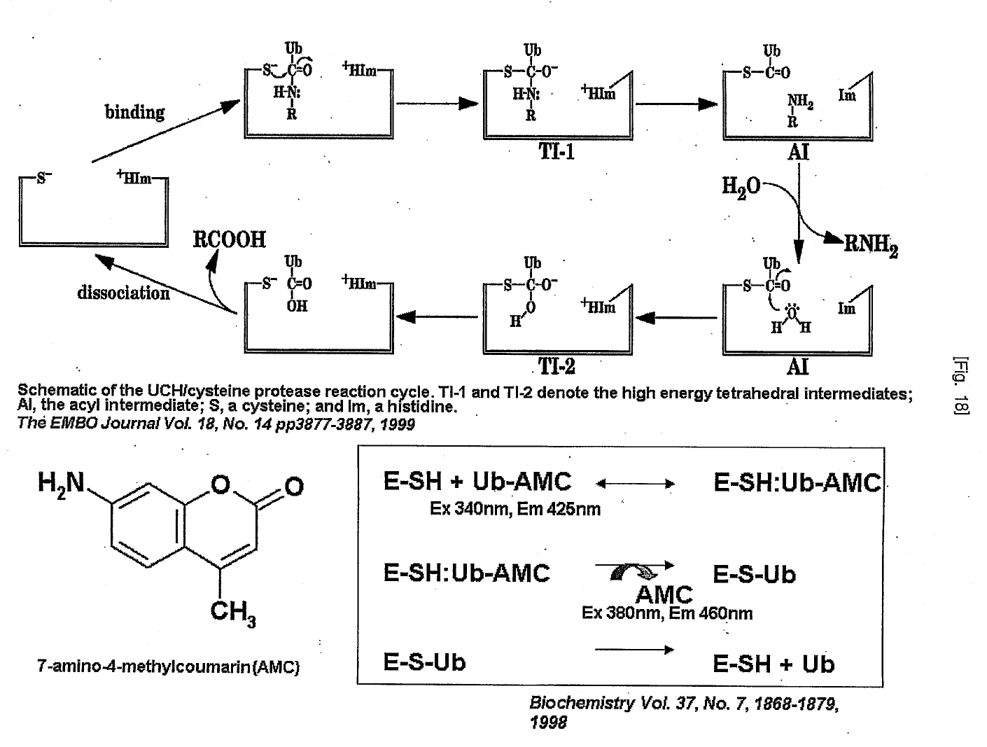

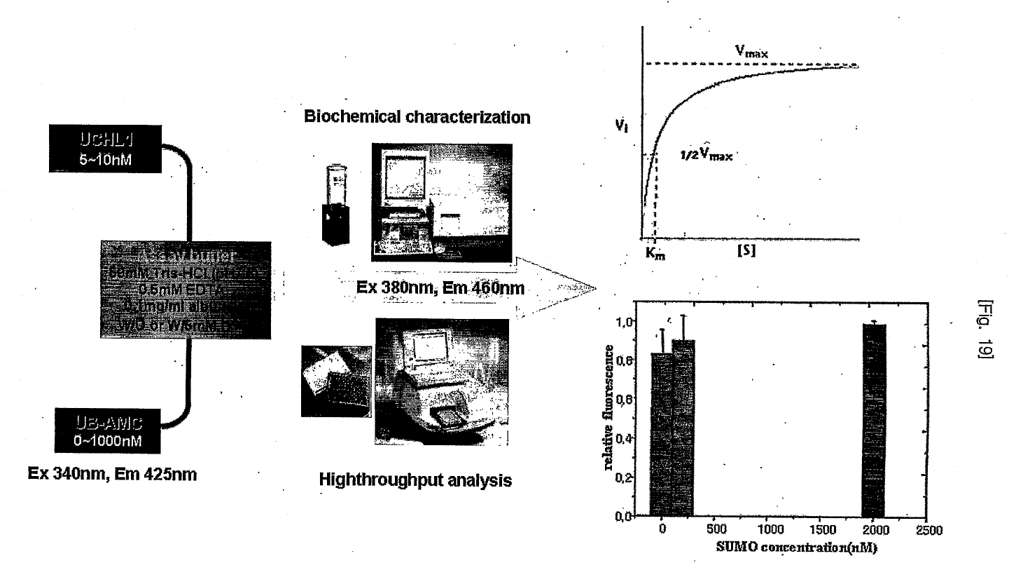

To screen the chemicals (inhibitors for cancer metastasis) to inhibit the expression or the hydrolase activity of UCH-L1, the high-throughput screening (HTS) can be used (FIGS. 18 and 19).

As shown in FIG. 18, the ubiquitin-AMC (Ub-AMC, 7-amino-4-methylcoumarin C-terminus derivative of ubiquitin) may be use as an artificial fluorogenic substrate for UCH-L1. The “hits” as inhibitors of UCH-L1 may be detected by monitoring the rate of release of free AMC (relative fluorescent intensity) from the Ub-AMC by UCH-L1.

In detail, when the mixture of UCH-L1 (5˜10 nM) and Ub-AMC (0˜1000 nM) is reacted with various candidate chemicals (various concentration) as inhibitors in UCH assay buffer (50 mM Tris-HCl [pH 7.6], 0.5 mM EDTA, 0.1 mg/mL albumin, with or without 5 mM of Dithiothreitol), the relative fluorescent intensities can be monitored to validate the hydrolase activity of UCH-L1 and the rate of UCH-L1 enzyme reaction using HTS (FIG. 19).

As stated above, UCH-L1 inhibitors obtained by using HTS may be used as a composition for suppression and prevention of cancer metastasis.

1. A composition for suppressing cancer metastasis comprising an inhibitor of UCH-L1.

2. The composition of claim 1, wherein the inhibitor of UCH-L1 is shRNAi of UCH-L1.

3. The composition of claim 2, wherein the shRNAi of UCH-L1 is one or more oligonucleotides selected from oligonucleotides of SEQ ID Nos. 8, 9 and 10.

4. The composition of claim 1, wherein the inhibitor of UCH-L1 is a monoclonal antibody for UCH-L1.

5. The composition of claim 1, wherein the inhibitor of UCH-L1 is a polyclonal antibody for 11 amino acids located in N-terminal region of UCH-L1.

6. A composition for screening a cancer metastasis inhibitor comprising UCH-L1 protein having the amino acid sequence of SEQ ID No.: 1.

7. A composition for screening a cancer metastasis inhibitor comprising UCH-L1 gene having the base sequence of SEQ ID No.: 2.

8. (canceled)

9. (canceled)

10. (canceled)

11. (canceled)

12. (canceled)

13. (canceled)

14. (canceled)

15. A method of suppressing cancer metastasis which comprises administrating to a subject a therapeutically effective amount of an inhibitor of UCH-L1.

16. The method of claim 15, wherein the inhibitor of UCH-L1 is shRNAi of UCH-L1.

17. The method of claim 16, wherein the shRNAi of UCH-L1 is one or more oligonucleotides selected from oligonucleotides of SEQ ID Nos. 8, 9 and 10.

18. The method of claim 15, wherein the inhibitor of UCH-L1 is a monoclonal antibody for UCH-L1.

19. The method of claim 15, wherein the inhibitor of UCH-L1 is a polyclonal antibody for 11 amino acids located in N-terminal region of UCH-L1.

20. A screening method of a cancer metastasis inhibitor consisting of contacting a test material to a composition for screening a cancer metastasis inhibitor comprising UCH-L1 protein having the amino acid sequence of SEQ ID No.: 1 as a target material, then examining their reaction, thereby deciding that the test material activates or suppresses the function of the protein.

21. A screening method of a cancer metastasis inhibitor consisting of contacting a test material to a composition for screening a cancer metastasis inhibitor comprising UCH-L1 gene having the base sequence of SEQ ID No.: 2 as a target material, then examining their reaction, thereby deciding that the test material activates or suppresses the expression of the gene.
